(19) United States
(12) Patent Application Publication
(10) Pub. No.: US 2017/0027881 A1
(43) Pub. Date: Feb. 2, 2017

(54) COMPOSITION AND RELATED METHODS FOR TREATMENT OF PILOSEBACEOUS DISEASES

(71) Applicant: Neil A. Shah, St. Paul, MN (US)

(72) Inventor: Neil A. Shah, St. Paul, MN (US)

(21) Appl. No.: 15/224,874

(22) Filed: Aug. 1, 2016

Related U.S. Application Data
(60) Provisional application No. 62/199,049, filed on Jul. 30, 2015.

(51) Int. Cl. A61K 31/045; A61K 9/00; A61K 45/06; A61K 31/047

(52) U.S. Cl. CPC A61K 31/045; A61K 31/047; A61K 9/0014; A61K 45/06

(57) ABSTRACT

Compositions and related methods of treating diseases of the pilosebaceous unit including acne, rosacea and hidradenitis. Through the introduction of active ingredients including xylitol and farnesol at a treatment location, biofilms associated with acne, roasacea and hidradenitis are disrupted and/or inhibited. The composition can be in the form of a topical medication or cream including a pharmaceutically acceptable carrier and the active ingredients. The composition can further comprise beneficial skin supplements to further enhance the treatment effects. The composition can be utilized in conjunction with additional biofilm disruption treatments.

### RELATED APPLICATION

The present application claims the benefit of U.S. Provisional Application No. 62/199,049 filed Jul. 30, 2015, which is hereby incorporated herein in its entirety by reference.

### FIELD OF THE INVENTION

The present invention is generally directed to the field of topically applied pharmaceuticals for treating diseases of the pilosebaceous unit. More particularly, the present invention is directed to a composition and related methods of treatment of the pilosebaceous unit using a topically applied cream comprises of xylitol and farnesol for disrupting biofilm at a treatment location.

### BACKGROUND OF THE INVENTION

Biofilms are protective aggregates of polysaccharides and/or lipopolysaccharides which are secreted by members of a bacterial colony. Biofilms serve to help protect the bacteria within from physical disruption, temperature changes, intrinsic immune system attacks and extrinsic antibiotics. Biofilms play a central role in the pathogenesis of many diseases including infected prosthetic devices, dental caries and common skin diseases including disorders of the pilosebaceous unit.

Acne, rosacea and hidradenitis are three common conditions that belong to the family of diseases of the pilosebaceous unit. The pathophysiology of these diseases is complex and multifactorial comprising both external and intrinsic factors. The most important extrinsic factor is bacterial growth. Bacteria, either free living (planktonic) or in colonies (biofilms), are thought to play an important role in these diseases either by frank infection or by stimulation of an aberrant auto-inflammatory response in the host. Although the current state of understanding of these diseases acknowledges the role of biofilms in these diseases, the degree to which biofilms are central to the pathophysiology of these diseases is unclear.

Currently, antibiotics and antimicrobials are common treatments for all of these diseases, either in topical or oral form. These treatments have only been partially helpful from a clinical standpoint. It is known that biofilms can dramatically reduce the effectiveness of antimicrobial and antibiotic compounds. Despite this knowledge there are no commercially available medications designed to disrupt the bacterial biofilms implicated in these diseases. In addition, there are concerns regarding worsening antibiotic resistance with our current treatments.

Many biofilm disruptors or inhibitors exist in the prior art both within and outside the field of medicine. For example, some known biofilm disruptors or inhibitors are used as excipients in other cosmetics/medications, as flavoring and fragrance agents, and as active ingredients in the prevention and treatment of other biofilm-mediated conditions such as, for example, dental caries. Burkhart published the first papers implicating biofilms in acne pathogenesis in 2003. However, since that time there has been little advancement in terms of effective treatments targeting biofilms in the pilosebaceous diseases. In fact, the relative novelty of the concept of treating the biofilm component of the pilosebacous diseases is borne out by the paucity of scientific literature regarding biofilms and acne, the most common of the pilosebaceous disorders. Out of 14,444 articles in PUBMED, a large searchable database of the scientific literature, related to ‘acne’ only 37 appear for the search terms ‘acne biofilm.’

As regards effective biofilm disruptors for cutaneous disease, the most pertinent prior art is a study published by Masako et at in 2005 (Journal of Dermatological Science, 38(3), June 2005, Pages 197-205) using a combination of xylitol and farnesol for the treatment of atopic dermatitis, also known as eczema. While eczema is a disease of the skin, it is wholly different in terms of pathophysiology, effective treatments, and even clinical appearance as to constitute an unrelated disorder to the diseases of the pilosebaceous unit. For example, topical corticosteroid formulations are effective for the treatment of eczema but would be harmful if used for the treatment of rosacea. In fact, since 2005 the only published references to these compounds in the medical literature have been related to chronic wounds and dentistry.

While a variety of treatments and protocols exist for treating the pilosebaceous unit, to date there has been nothing developed to address biofilms and their role in the treatment of acne, rosacea and hidradenitis. As such, it would be advantageous to have a composition capable of disruption and/or inhibiting biofilms associated with these diseases.

### SUMMARY OF THE INVENTION

The present invention is directed to a topically applied medication for disrupting and/or inhibiting biofilms associated with acne, rosacea and hidradenitis. Generally, the composition comprises a pharmaceutically acceptable delivery system including the active ingredients xylitol and farnesol. Preferably, the pharmaceutically acceptable delivery system comprises a topical medication or cream that can be applied directly to a treatment location. In some embodiments, the composition can comprise additional active ingredients that target bacteria including, for example, vitamin A-related compounds, antimicrobial compounds, antibiotic compounds and combinations thereof. In addition to the active ingredients, in some embodiments, the composition can comprise a variety of other components having beneficial skin properties such as, for example, antioxidants, moisturizers, cleansers, vitamins, ultraviolet protection and the like. In some embodiments, the topically applied medication can be used in conjunction with additional biofilm disrupting and/or inhibiting treatments including, for example, ultrasound, radiofrequency, thermal treatments, laser treatments and combinations thereof.

In one representative aspect, the present invention comprises a topical medication for the treatment of diseases of the pilosebaceous unit including acne, rosacea and hidradenitis. Generally, the topical medication comprises a pharmaceutically acceptable carrier and active ingredients including xylitol and farnesol for disrupting and/or inhibiting biofilm associated with pilosebaceous diseases. Generally, the topical medication can comprise the active ingredients in individual amounts ranging from about from 0.0001 to about 50% by weight and wherein the combination of the active ingredients comprise from about 0.0002 to about 100% by weight of the topical medication. In some embodiments, the topical medication can comprise additional bacteria targeting active ingredients such as, for example, vitamin A-related compounds, antimicrobial compounds, antibiotic compounds and combinations thereof. The topical medication can further comprise additional supplements that can be selected as having desirable skin application characteristics including, for example, antioxidants, moisturizers, cleansers, vitamins, ultraviolet protection and the like as well as combinations thereof. In some embodiments, the topical medication can comprise an over-the-counter product wherein in other embodiments, the topical medication can comprise a prescription strength medication. In some embodiments, the topical medication can be used in conjunction with additional biofilm disrupting and/or inhibiting treatments including, for example, ultrasound, radiofrequency, thermal treatments, laser treatments and combinations thereof.

In another aspect, the present invention can comprise a method for treating diseases of the pilosebaceous unit including acne, rosacea and hidradenitis. Generally, the method can comprise a step of delivering active ingredients including xylitol and farnesol to a treatment location. The method can further a step of disrupting and/or inhibiting a biofilm at the treatment location. In some embodiments, the step of delivering can further comprise applying a topical cream at the treatment location, wherein the topical cream comprises a pharmaceutically acceptable carrier and the active ingredients. In some embodiments, the step of applying the topical cream can further comprise administering the active ingredients wherein the amounts of xylitol and farnesol individually range from about from 0.0001 to about 50% by weight and wherein the combination of the active ingredients comprises from about 0.0002 to about 100% by weight of the topical medication. In some embodiments, the method can further comprise targeting bacteria at the treatment location through the inclusion of additional bacteria-targeting active ingredients in the topical cream such as, for example, vitamin A-related compounds, antimicrobial compounds, antibiotic compounds and combinations thereof. In some embodiments, the method can further comprise supplementing the active ingredients with components having beneficial skin characteristics including, for example, antioxidants, moisturizers, cleansers, vitamins, ultraviolet protection and the like. In some embodiments, the method can further comprise providing the topical medication as an over-the-counter product wherein in other embodiments, the method can comprise providing the topical medication as a prescription strength medication. In some embodiments, the method can further comprise using additional biofilm disruption and/or inhibition steps including treatments such as, for example, ultrasound, radiofrequency, thermal treatments, laser treatments and combinations thereof in conjunction with applying the topical cream.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE DRAWINGS

The invention can be completely understood in consideration of the following detailed description of various embodiments of the invention in connection with the accompanying drawings, in which:

FIG. 1 is a chemical structure of xylitol according to the prior art.

FIG. 2 is a chemical structure of farnesol according to the prior art.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

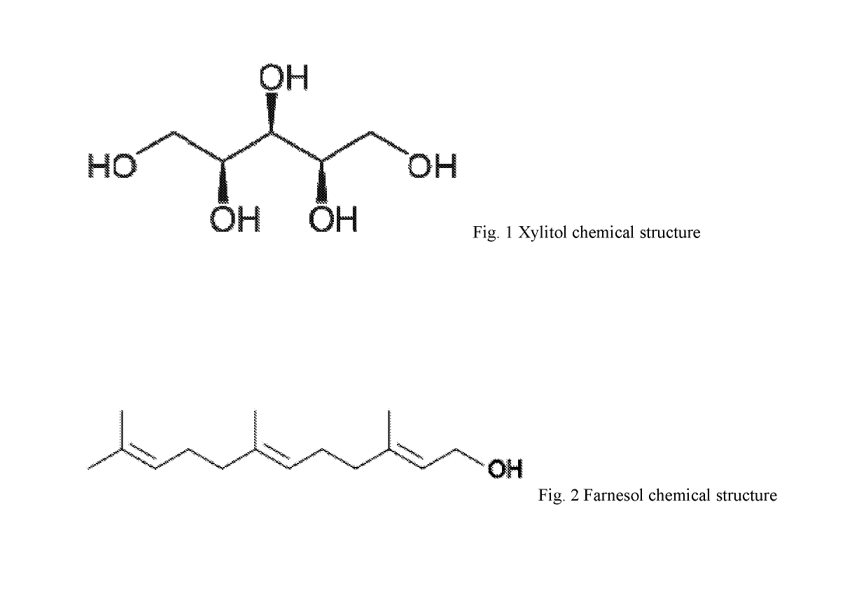

The present invention is directed to the use of active ingredients including xylitol and farnesol in the treatment of diseases of the pilosebaceous unit including acne, rosacea and hidradenitis. Xylitol, as depicted in FIG. 1, and farnesol, as depicted in FIG. 2, are innocuous in terms of potential for harm to the body. Xylitol is classified as ‘generally regarded as safe’ (GRAS) for cosmetic formulations by the FDA and is conventionally utilized in consumer products such as, for example, toothpastes and chewing gum. Farnesol is also classified as ‘generally regarded as safe’ by the FDA and is an ingredient commonly found in fragranced products. By treating diseases of the pilosebaceous unit with GRAS substances such as xylitol and farnesol, the potential for toxicity that is found with current treatments such as antibiotics is avoided.

Generally, the active ingredients of the present invention can be delivered to a treatment location as part of a topical medication. The topical medication can comprise a pharmaceutically acceptable carrier and the active ingredients including xylitol and farnesol, wherein the xylitol and farnesol disrupt and/or inhibit biofilm associated with pilosebaceous diseases at the treatment location. Generally, the topical medication can comprise the active ingredients in individual amounts ranging from about from 0.0001 to about 50% by weight and wherein the combination of the active ingredients comprise from about 0.0002 to about 100% by weight of the topical medication. In some embodiments, the topical medication can comprise additional bacteria targeting active ingredients such as, for example, vitamin A-related compounds, antimicrobial compounds, antibiotic compounds and combinations thereof. The topical medication can further comprise additional components that can be selected as having desirable skin application characteristics including, for example, antioxidants, moisturizers, cleansers, vitamins, ultraviolet protection and the like. In some embodiments, the topical medication can comprise an over-the-counter product wherein in other embodiments, the topical medication can comprise a prescription strength medication. In some embodiments, the topical medication can be used in conjunction with additional biofilm disrupting and/or inhibiting treatments including, for example, ultrasound, radiofrequency, thermal treatments, laser treatments and combinations thereof.

In treating diseases of the pilosebaceous unit (acne, rosacea and hidradenitis), the active ingredients disrupt and/or inhibit the formation of biofilms at a treatment location, whereby bacteria at the treatment location is no longer protected from immune responses and other healing mechanisms. Generally, treatment is accomplished through the delivery of active ingredients including xylitol and farnesol to the treatment location. In some embodiments, delivery can be accomplished through application of a topical medication or cream directly to the treatment location. The topical medication or cream generally comprises a pharmaceutically acceptable carrier and the active ingredients. Application of the topical medication or cream can further comprise administering the active ingredients of xylitol and farnesol in amounts that individually range from about 0.0001 to about 50% by weight of the topical medication and wherein the combination of the active ingredients comprises from about 0.0002 to about 100% by weight of the topical medication. The method can further comprise can comprise applying additional bacteria targeting active ingredients such as, for example, vitamin A-related compounds, antimicrobial compounds, antibiotic compounds and combinations thereof as part of the topical medication or cream. The topical medication or cream can comprise additional beneficial supplements including, for example, antioxidants, moisturizers, cleansers, vitamins, ultraviolet protection and the like, to further enhance the beneficial effects of the topical medication. In some embodiments, the topical medication or cream can be available as an over-the-counter product wherein in other embodiments, the topical medication or cream can be made available as a prescription strength medication. In some embodiments, the method can comprise additional biofilm disruption and/or inhibition steps including, for example, ultrasound treatment, radio frequency treatment, thermal treatments, laser treatments and combinations thereof.

Preliminary in vivo case studies of vehicle base (control) versus xylitol/farnesol in vehicle base (experimental) show a reduction in the number of inflammatory rosacea papules on the experimental side.

### Example

### Patient Study—Xylitol 5%+Farnesol 0.2% in an Emollient Base

A proof-of-concept, open-label, uncontrolled, nonrandomized trial was designed to obtain a preliminary assessment of the efficacy of the active ingredients of the present invention. A total of eleven (11) consecutive patients (10 female, 1 male; mean age 49) with papulopustular (type II) rosacea who were treatment naïve and not currently using any other topical medications on their face were recruited. Informed consent was obtained in accordance with the principles of the Declaration of Helsinki. Instructions for skin care were provided to each of the patients; they were instructed to not use any soaps, medications, moisturizers, cosmetics or other topical preparations on the face while using the topical medication of the present invention.

The topical medication was prepared using a bland emollient base (study cream) as the pharmaceutically acceptable carrier. The active ingredients were added to the study cream in an amount such that xylitol constituted 5% by weight of the topical medication with farnesol constituting 0.2% by weight of the topical medication. The topical medication was provided to each of the patients with the same instructions to apply the topical medication twice daily to their entire face. Following 30 days of continual treatment with the topical medication, the patients were assessed in person. As part of the assessment, each patient was asked to evaluate the performance of the topical medication by rating any improvement in their condition as compared to their baseline condition prior to treatment with the topical medication. Patients were asked to rate their improvement from the baseline condition as one of: none, minimal, modest, or excellent.

Based on the patient assessments, a large majority of the patients experienced improvement to their papulopustular (type II) rosacea following 30 days of treatment with the topical medication of the present invention. Nine (9) of the patients reported their improvement as “excellent”. One “1” patient rated their improvement as “modest”. Only one “1” patient reported their improvement as “none”. Just as importantly, there were no reports of any side effects as a result of utilizing the topical medication. As such, the topical medication of the present invention was safely utilized to provide some level of improvement in over 90% of the trial group with their papulopustular (type II) rosacea.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A topical medication for treating diseases of the pilosebaceous unit, comprising:
a biofilm disruptor including active ingredients, said active ingredients including xylitol and farnesol; and
a pharmaceutically acceptable carrier.

2. The topical medication of claim 1, wherein the active ingredients range from about 0.0002 to about 100 percent by weight of the topical medication.

3. The topical medication of claim 2, wherein xylitol is present in an amount from about 0.0001 to about 50 percent by weight of the topical medication.

4. The topical medication of claim 2, wherein farnesol is present in an amount from about 0.0001 to about 50 percent by weight of the topical medication.

5. The topical medication of claim 1, wherein xyltol is present in an amount of 5% by weight of the topical medication and farnesol is present in an amount of 0.2% by weight of the topical medication.

6. The topical medication of claim 5, wherein the pharmaceutically acceptable carrier comprises a bland emollient base.

7. The topical medication of claim 1, wherein the active ingredients further comprise:
bacteria targeting ingredients.

8. The topical medication of claim 7, wherein the bacteria targeting ingredients are selected from the group consisting essentially of: vitamin A-related compounds, antimicrobial compounds, antibiotic compounds and combinations thereof.

9. The topical medication of claim 1, further comprising a beneficial skin supplement.

10. The topical medication of claim 9, wherein the beneficial skin supplement is selected form the group consisting essentially of: moisturizers, cleansers, vitamins, ultraviolet protection and the like and combinations thereof.

11. A method for treating pilosebaceous diseases, comprising:
disrupting biofilm at a treatment location through the application of a topical medication having active ingredients, the active ingredients including xylitol and farnesol.

12. The method of claim 11 wherein the pilosebaceous disease is selected from the group consisting essentially of acne, rosacea and hidradenitis.

13. The method of claim 11, further comprising:
preparing the topical medication, wherein the topical medication comprises a pharmaceutically acceptable carrier and the active ingredients.

14. The method of claim 13, wherein the active ingredients are present in an amount from about 0.0002 to about 100 percent by weight of the topical medication.

15. The method of claim 14, wherein the xylitol is present in an amount from about 0.0001 to about 50 percent by weight of the topical medication.

16. The method of claim 14, wherein the farnesol is present in an amount from about 0.0001 to about 50 percent by weight of the topical medication.

17. The method of claim 13, further comprising:
treating bacteria at the treatment location with a bacteria-targeting active ingredient included within the topical medication.

18. The method of claim 17, wherein the bacteria-targeting active ingredient is selected from the group consisting essentially of: vitamin A-related compounds, antimicrobial compounds, antibiotic compounds and combinations thereof.

19. The method of claim 13 wherein the topical medication further comprises a beneficial skin supplement.

20. The method of claim 19, wherein the beneficial skin supplement is selected from the group consisting essentially of: moisturizers, cleansers, vitamins, ultraviolet protection and combinations thereof.

21. The method of claim 13, further comprising:
utilizing a supplemental biofilm disruption treatment in conjunction with application of the active ingredients to the treatment site.

22. The method of claim 21, wherein the supplemental biofilm disruption treatment is selected from the group consisting essentially of: ultrasound treatments, radio frequency treatments, thermal treatments, laser treatments and combinations thereof.
